(19) United States
(12) Patent Application Publication
(10) Pub. No.: US 2005/0090812 A1
(43) Pub. Date: Apr. 28, 2005

(54) Surgical device and method of use

(76) Inventor: John H. Shadduck, Tiburon, CA (US)

Correspondence Address: John H. Shadduck, 1490 Vistazo West, Tiburon, CA, 94920, US

(21) Appl. No.: 10/692,961

(22) Filed: Oct. 24, 2003

(51) Int. Cl. A61B 18/18

(52) U.S. Cl. 606/2; 607/88

(57) ABSTRACT

A medical device having a working surface of a photonic lattice for controlled diffraction of electromagnetic energy within, and energy emissions from, the working surface to control energy delivery to tissue. The working surface can apply energy to tissue at high or low intensities for thermal therapies, ablations or volumetric removal of tissue volumes. In one embodiment, the energy emitting surface comprises a lattice of a refractory material with interior spatial regions of a selected geometry to provide a band gap. The energy modes confined within the lattice can create a high intensity conditioned plasma for delivering energy to tissue positioned proximate to the lattice. In an exemplary embodiment, the photonic lattice defines a lattice constant of less than about 5 microns for altering a non-preferred energy mode to a preferred mode to control infrared emissions from the working surface. Additional Rf energy can be coupled to the conditioned plasma for enhanced application of energy to tissue.

### BACKGROUND OF THE INVENTION

1. Field of the Invention

The present invention relates to medical devices and more particularly to a working surface for applying energy to tissue that utilizes a refractory photonic lattice for diffraction and control of energy emissions from the working surface to interact with tissue.

2. Description of the Related Art

In recent years, theoretical and experimental work has been undertaken in the field of photonic lattices. The experiments have been directed toward creating photonic bandgaps in various wavelength bands in periodic crystalline solid materials or lattices that have open spatial geometries. Photonic lattices can be designed to control and redirect the propagation of light without energy loss. In one early experiment relating to photonic lattices, Yablonovitch et al. (E. Yablonovitch. Phys. Rev. Lett., 58, 2059 (1987)) concluded that electromagnetic radiation propagating in periodic dielectric structures is similar to electron waves propagating in a crystal. Yablonovitch et al. postulated that periodic refraction patterns in a lattice would create a band structure for electromagnetic waves wherein particular wavelengths either propagate or cannot propagate. In periodic structures in optical wavelength dimensions, a photonic bandgap would exist, i.e., a frequency range in which photons are not allowed to propagate. Such photonic lattices could exist in two or three dimensions—and result in phenomena such as inhibition of spontaneous emission from an atom that radiates inside the photonic gap or frequency selective transmission and reflection. These properties of photonic lattices would allow the guiding and filtering of light as it propagates within the lattice. In one example, a photonic lattice could be constructed to provide a full photonic bandgap, i.e., a photonic insulator that is created by artificial control the optical properties of the solid (lattice).

### SUMMARY OF THE INVENTION

In general, the present invention relates to medical device working ends for applying energy to tissue for thermal therapies, ablations or volumetric removal of tissue volumes. More particularly, the invention for the first time provides an energy emitting surface comprising a photonic lattice with interior spatial regions of various geometries for controlling thermal emissivity and/or for creating and controlling plasma about the lattice for applying energy to tissue.

In an exemplary instrument, the working surface carries a photonic lattice that defines a dimensional constant in the 4.0 to 4.4 micron range that will provide a photonic bandgap in the infrared wavelength range. The lattice is fabricated of a refractory material so that it will function to heat the lattice and emit wavelengths in the infrared. The spatial geometry of the lattice will confine these modes within the lattice, alter the modes that can emit from the working surface, and create an high energy plasma within and about the lattice. By painting the working surface over tissue or placing the working surface in proximity to tissue-whether in an underwater surgery or in a dry surgery—the surface will apply ablative energy to the tissue surface. Several photonic bandgap structures or refractory lattices are described which can alter energy modes in the lattice for controlling energy particle emissions from the working surface or controlling particle trajectories to create a conditioned plasma about the lattice surface for applying energy to tissue.

In general, the invention advantageously provides a medical instrument with a working surface of a photonic lattice for controlling emissions therefrom.

The invention provides an electrosurgical working surface of a refractory photonic lattice that alters optical modes.

The invention advantageously provides a working surface of photonic lattice that defines a plurality of spatial regions therein that act as diffraction centers for energy particles.

The invention provides a photonic lattice that allows for controlled diffraction of energy particles about the lattice and working surface to condition a plasma for ablative interaction with tissue.

The invention provides an instrument working surface of refractory lattice that produces a high energy plasma for ablative interaction with tissue.

The invention provides an instrument working surface of lattice that allows for practically 100% engagement of a tissue surface with an ionized gas for uniform coupling of electrical energy to tissue.

The invention provides an instrument working surface of photonic lattice that alters optical modes from a longer infrared wavelength to a shorter wavelength.

These and other objects and features of the present invention will become readily apparent upon further review of the following drawings and specification.

### BRIEF DESCRIPTION OF THE DRAWINGS

The accompanying drawings are incorporated into and form a part of this specification, and illustrate the present invention together with the description of the invention. In the drawings, like elements are referred to by like reference numerals.

FIG. 1 is a schematic illustration of a portion of a Type “A” three-dimensional photonic lattice with an ordered periodicity for use in a medical device working surface.

FIG. 2 is an enlarged sectional illustration of a photonic lattice as in FIG. 1 carried in a working surface of a medical probe.

FIG. 3 is a sectional view of a probe's working end with a photonic lattice carried in the working surface.

FIG. 4 is a perspective view of an alternative probe with a photonic lattice wire element.

FIG. 5 is a schematic illustration of an alternative Type “A” three-dimensional photonic lattice with an ordered periodicity and a differentiated surface layer.

FIG. 6A is a schematic illustration of a first step in a method of using an exemplary three-dimensional photonic lattice for applying energy to tissue.

FIG. 6B is a schematic illustration of the confinement of an infrared band within the lattice to create a conditioned plasma about the lattice wherein energy particles are diffracted and organized to interact with tissue positioned near the lattice.

FIG. 7 is a schematic illustration of a Type “B” three-dimensional lattice of a refractory material with surface layers having an electrically insulated coating.

FIG. 8 is an illustration of an alternative three-dimensional lattice of a refractory material with non-uniform lattice constants for selective confinement and emission of particular energy bands.

### DETAILED DESCRIPTION OF THE INVENTION

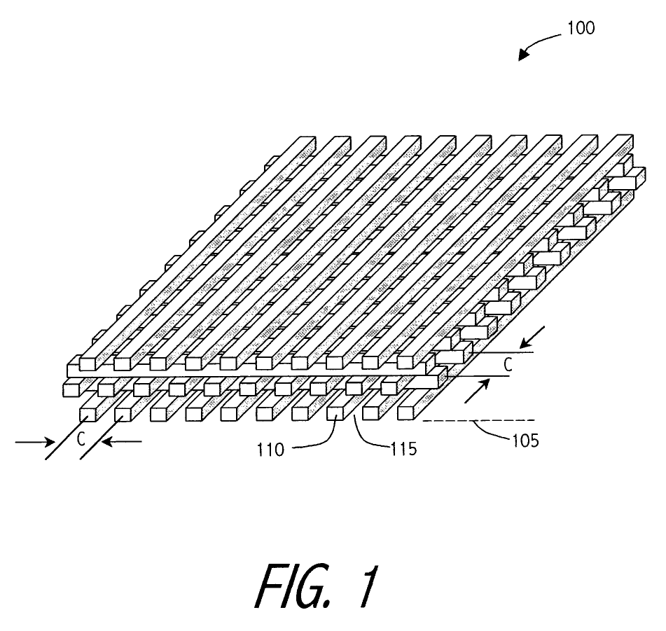

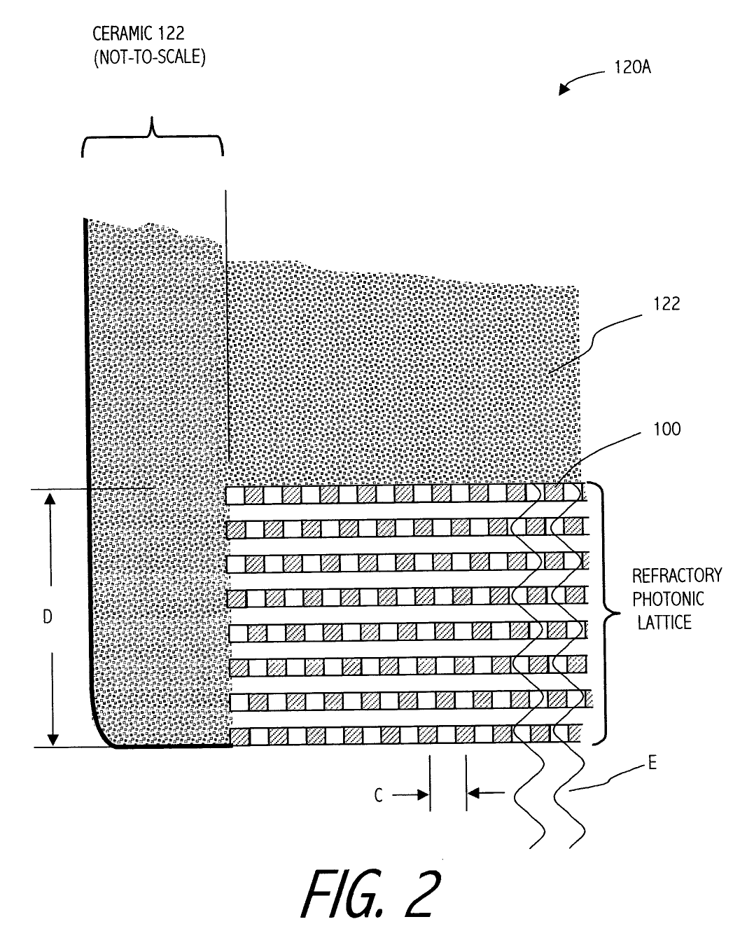

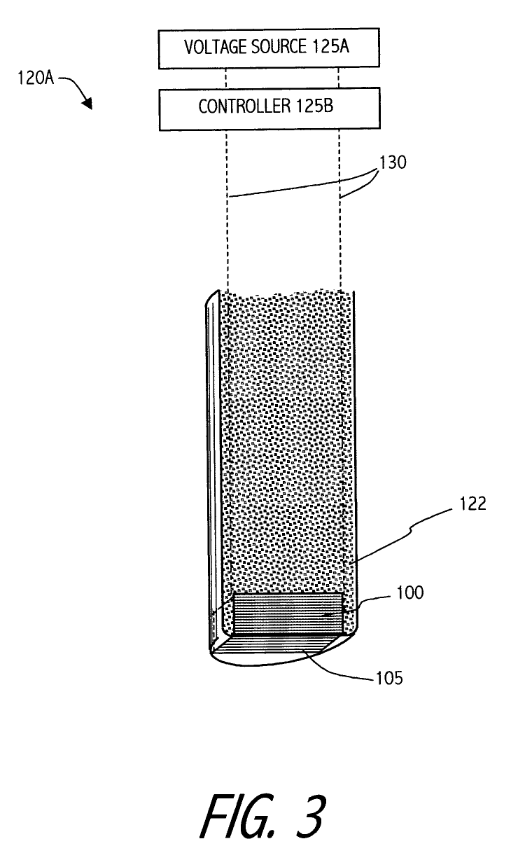

1. Type “A” medical device with photonic lattice for controlling optical modes. The present invention comprises a surgical instrument with a working end for applying energy to biological structures for ablation and volumetric removal procedures. FIG. 1 is a perspective schematic illustration of a three-dimensional photonic lattice structure 100 that is configured as an energy emitter in a medical instrument (see FIGS. 2 and 3). The use of such two dimensional or three dimensional photonic lattices is proposed in a surgical method: for controlling, directing and harmonizing energy particle propagation within and about the lattice-emitter which is carried in a medical device's working surface 105. The two or three dimensional photonic lattice defines a structure of a first dielectric material 110 with a second dielectric material in spatial regions 115 therein that act as diffraction centers for energy particles. In one exemplary embodiment, the photonic lattice defines a lattice constant C in the range of about 4.0 to 4.4 microns to provide a photonic bandgap covering the infrared wavelength range (see FIG. 1). The rod-like elements of the lattice can have dimensions in the 0.8 to 2.5 micron range. In another embodiment, for example, the lattice can define non-uniform dimensions to provide a more precise photonic bandgap within the infrared band to permit certain infrared wavelength emissions while confining other infrared wavelengths. Various photonic bandgap structures in the infrared and visible bands, and bandgap strategies, can provide varied means (i) for controlling energy particle emissions from the working surface, (ii) for controlling energy particle trajectories in plasmas confined within the lattice and about the lattice's exposed surface, (iii) for controlling ions to create a harmonious plasma for coupling electrical energy to tissue, (iv) for controlling energy particle propagation to selectively convert energy bands from a first mode to a second mode, and (v) for controlling and guiding energy emissions within, and from, the lattice. The scope of the invention encompasses the use of such photonic lattice means for controlling energy-emissions and propagations in surgical uses, and includes the use of ordered and disordered microfabricated lattices in medical device working surfaces. As used herein, the term energy particles refers to electromagnetic waves, light particles or photons, electons, ions, microwaves, magnetic waves, elastic waves and the like. For example, an electromagnetic wave is indicated at E in FIG. 2 having a wavelength that can propagate in the second dielectric or spatial geometry of the lattice. When describing an ionized gas within a photonic lattice, the terms harmonious, conditioned and the like are used herein to describe a plasma in which energy particles movements are controlled, ordered and less chaotic than if not contained within ordered wavelength scale spatial geometries as in lattice 100.

An exemplary photonic lattice as in FIGS. 1 and 2 comprises a structure of first and second dielectric materials or media 110 and 115, respectively, with a periodic variation in the first and second media on the order of the wavelength of light. Such periodic variations will modify allowed optical modes within the structure, and emissivity from the structure. Such a photonic structure that completely eliminates optical modes in all directions for a specific wavelength band is often referred to as a band gap structure. These structures or lattices can exhibit a three-dimensional (3D) or a two-dimensional photonic band gap. The academic literature contains descriptions of such photonic lattices and their properties (see, e.g., Joannopoulos et al., Photonic Crystals: Molding the Flow of Light (1995)). The author's proposed use of such photonic lattices and band gap structures, it is believed, is the first in which the thermal emission spectrum is controlled and mode-altered to provide a conditioned plasma for allowing a new class of energy delivery in medical applications.

The manner of fabricating a photonic structure of a metallic material for use in incandescent emitter and similar uses is described in U.S. Pat. Nos. 6,611,085 and 6,583,350 to Gee et al., and U.S. Patent application publication no. 20030132705 to Gee et al, the complete disclosures of which are incorporated herein by reference. Lin et al. described the modification of thermal radiation from a photonic structure in the infrared spectrum, for example for use in light bulbs, in “Enhancement and suppression of thermal emission by a three-dimensional photonic crystal,” Phys. Rev B62, R2243 (2000).

FIGS. 2 and 3 illustrate a Type “A” working end 120A of a medical probe with a working surface 105 for engaging or positioning proximate to targeted tissue. The sectional thickness dimension D of the photonic lattice 100 as in FIG. 2 can be from as few as about 4 to 6 layers (i.e., 4×C to 6×C) of the ordered structure to any a larger number of layers. For the uses described herein, the lattice constant C is less than about 10 microns. More preferably, the lattice constant is less that about 5 microns. The lattice structure has a second dielectric or spatial region that comprises 50% of the volume of the lattice of FIGS. 1 and 2. Other ordered and disordered lattices can have from about 40% to about 80% open spatial portions and fall within the scope of the invention. An exemplary photonic lattice 100 is carried in a working end of an insulative material indicated at 122 (FIGS. 2-3). The working end 120A of FIGS. 2-3 comprises a probe end but it should be appreciated that the working end can be a blunt probe, needle-like or sharp probe, a blade-like edge, a planar surface, a jaw surface or a surface of an articulating member or catheter working end as is known in the art. The working surface can be as found in comparable classes of surgical instruments that utilize working ends for applying Rf or microwave energy to tissue. In the probe end of FIGS. 2 and 3, the photonic lattice 100 can have any surface area and is depicted as having a cross-sectional depth D of about 15 layers.

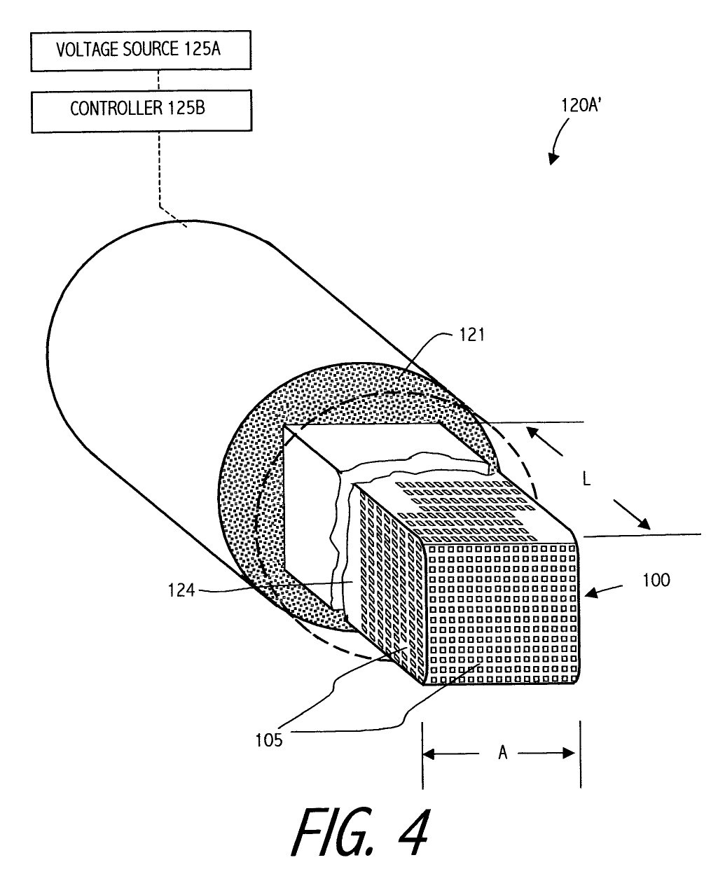

An alternative working end 120A′ is shown in FIG. 4 wherein the instrument has an insulated shaft portion 121 that extends to a photonic lattice 100 that comprises working surface 105 on all sides of a wire-like element 124. The small cross-section wire-lattice 100 of FIG. 4 is adapted for tissue cutting with a conditioned plasma, for example, for use in precision ophthalmic and neurosurgery applications. Such a wire can be in the range of 20 microns to 200 microns in cross-section A across a principal axis and have any length L. The wire is coupled to a voltage source 125A and controller 125B as shown in FIG. 4. In the wire-lattice embodiment of FIG. 4, the lattice 100 can be of a refractory (resistive) material wherein the electrical source 125A is adapted to resistively heat the lattice as described in greater detail below. Alternatively, the lattice 100 as in FIG. 4 can function as an active conductor or first polarity electrode element coupled to source 125A that comprises a radiofrequency generator. In operation, the small diameter wire-lattice will heat instantly upon Rf flow thought the lattice to create a conditioned plasma (as described below) within the lattice 100. In this embodiment, the second opposing polarity element (or return electrode) can be a ground pad or a conductor in the insulated shaft portion 121 that is coupled to source 125A.

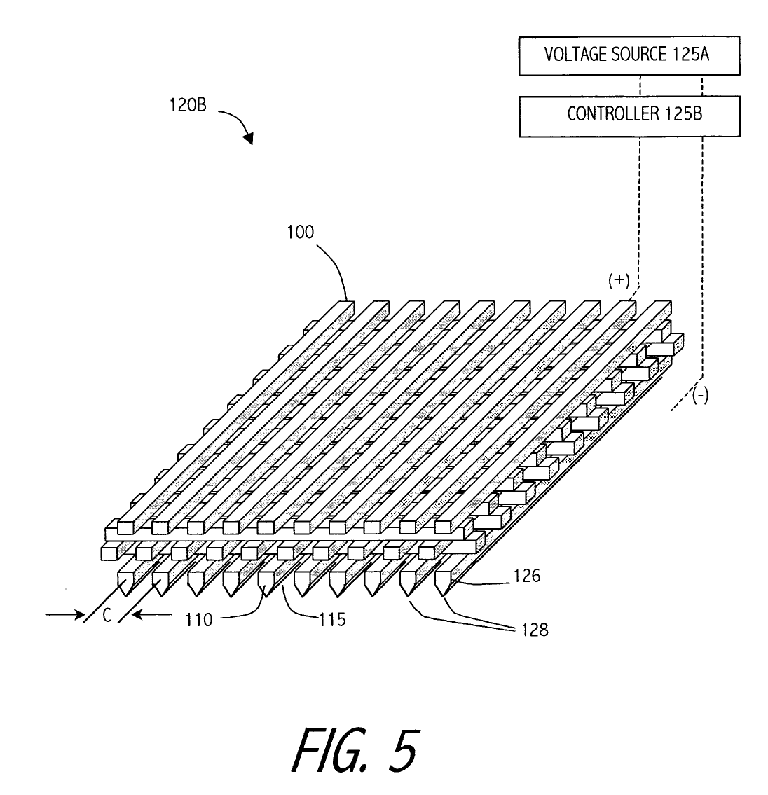

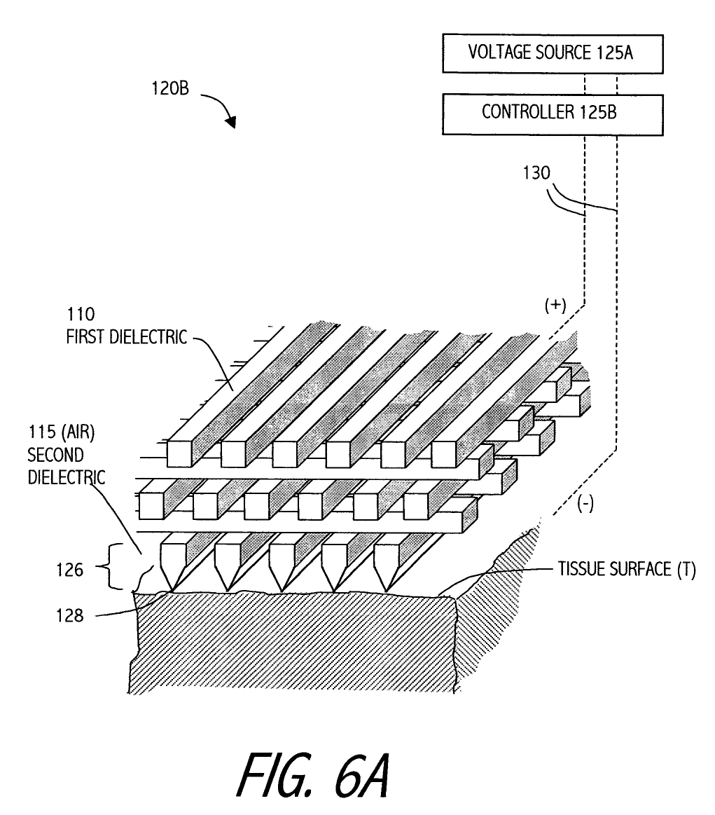

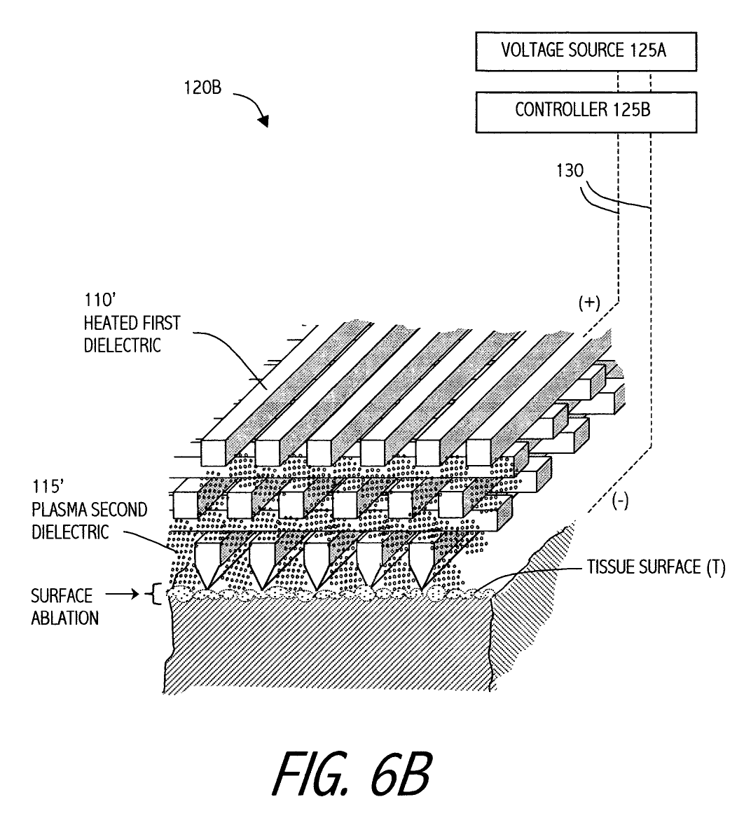

FIGS. 5, 6A and 6B schematically depict one Type “A” embodiment and a method of applying energy to tissue. In FIG. 5, the exemplary probe working end 120B can be any suitable shape and cross-section, for example from 0.1 mm to about 5 mm. The working surface 105 in FIG. 5 carries an exemplary photonic lattice 100 that is microfabricated of a refractory material such and tungsten, a tungsten alloy, a high temperature alloy of a transition metal or the like. The lattice also can be any refractory composition of any advanced materials under development that are combinations of metals and ceramics (see, e.g., Onnex™ by Excera Materials Group, Inc., 1275 Kinnear Road, Columbus, Ohio 43212).

Still referring to FIG. 5, in a small diameter embodiment, the working end 120B is adapted for precise ablative cutting, for example in ophthalmologic and neurological procedures. The introducer portion and handle (not shown) is of any suitable insulative material. The handle end is typically suited for gripping a human hand, but also includes any configuration for holding by robotic means, stereotactic positioning means and the like.

In the exemplary lattice embodiment of FIG. 5, the lattice 100 consists of a lattice structure having a uniform periodicity with a complete band gap in the infrared region. As can be seen in FIG. 5, one preferred (but optional) lattice embodiment has a surface layer 126 of the first dielectric 110 in the from of bar elements with a projecting edge indicated at 128. The photonic lattice has a first dielectric 110 of a refractory material (e.g., a tungsten alloy) that can be resistively heated or conductively heated and is adapted for instant thermal ionization of gas media within the open portion of the lattice. The lattice controls the optical modes to prevent infrared emissions from the working surface which causes the captured energy within the lattice to instantly ionize gas in the open spatial regions. The creation of a controlled, conditioned and harmonious plasma within the spatial regions 115 (second dielectric) of the lattice can be used to effectively apply energy to tissue proximate to the working surface 105. In one embodiment, the voltage source 125A and controller 125B are coupled to the lattice 100 by first and second (+) and (−) leads 130 (shown collectively in schematic diagram of FIGS. 5, 6A-6B). Now referring to FIG. 6B, a selected power level of electrical current flow from source 125A is used to instantly elevate the temperature of the refractory element now indicated at 110′. The altered state first dielectric 110′ (at a selected temperature between 100° C. and about 800° C. (not limiting)) in turn creates a plasma (altered second dielectric) indicated at 115′. In one embodiment, the lattice prevents emissions in the infrared band, which modeling suggests will create a high intensity conditioned plasma instantly with very low power levels that confines energy within and about the lattice. The ionized spatial region or plasma about the lattice surface (see FIG. 6B) will itself deliver energy to tissue surface T. Of particular interest, as shown in FIGS. 6A-6B, the tissue surface T is practically 100% engaged by such a conductive plasma due to the sharp edges 128 of the engagement surface. In an enhanced energy application means, the system can couple Rf energy to the tissue surface via the conductive gas 115′ from voltage source 125A. In this embodiment, the second dielectric 115′ functions, in effect, as a conductive gas electrode and can deliver very high energies in a continuous plane over the tissue surface T from source 125A (e.g., Rf generator). This energized gas electrode or second dielectric 115′ comprises a plasma that can ablate tissue by molecular volatilization resulting in volumetric removal ratably layer by layer as indicated in FIG. 6B. In such a lattice embodiment of FIGS. 5, 6A and 6B, the refractory material of the lattice is selected to have higher electrical resistance than any plasma therein so that current will naturally flow to the tissue via the gas electrode in continuous contact with the tissue.

In FIG. 6B, it should be appreciated that the tissue surface T can be “underwater” as in an arthroscopic procedure or on the surface of practically any body structure. In any arthroscopic cases, the fluid in the operating environment may enter the lattice but will be vaporized instantly. In a dry operating environment, atmospheric moisture will be present and will be vaporized upon energy delivery to the lattice. Such moisture in a fluid operating environment will contribute ions to the plasma that is sustained within and about the lattice 100. As can be seen if FIG. 6B, a return electrode is indicated at (−) which can be a ground pad or a return electrode on or about the working end or working surface of the instrument, any of which will couple with body media or the plasma to complete the electrical circuit. The return electrode also can be a similar lattice that creates a conductive gas electrode in a cooperating portion of the working end.

Thus, one method of the invention comprises providing a photonic lattice that is at least in part of a refractory material, elevating the temperature of a lattice portion by any means to thereby create an energetic plasma within and about the surface of lattice, and engaging the plasma with tissue to there by cause a controlled ablative energy-tissue interaction.

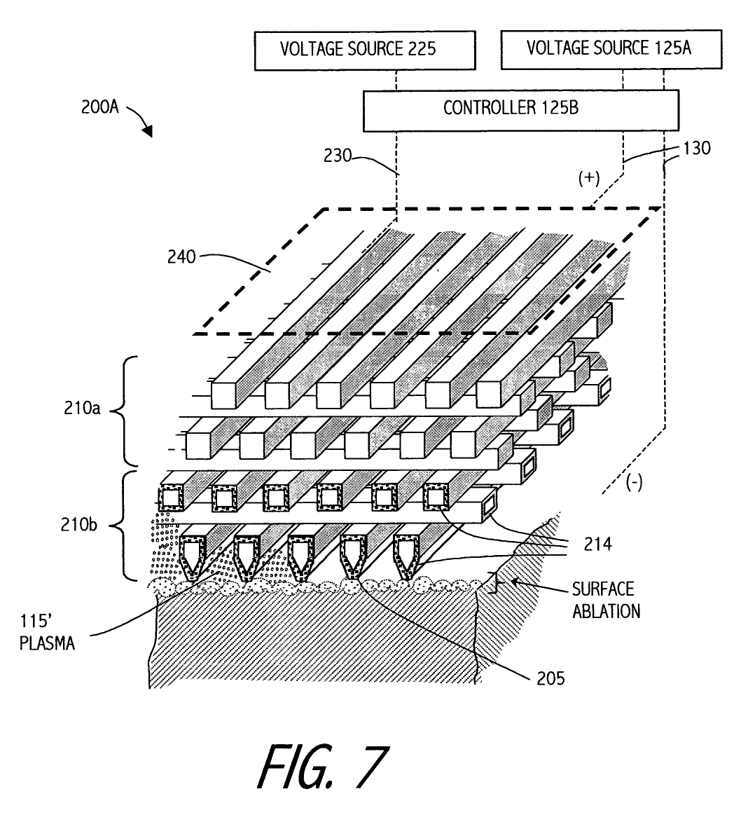

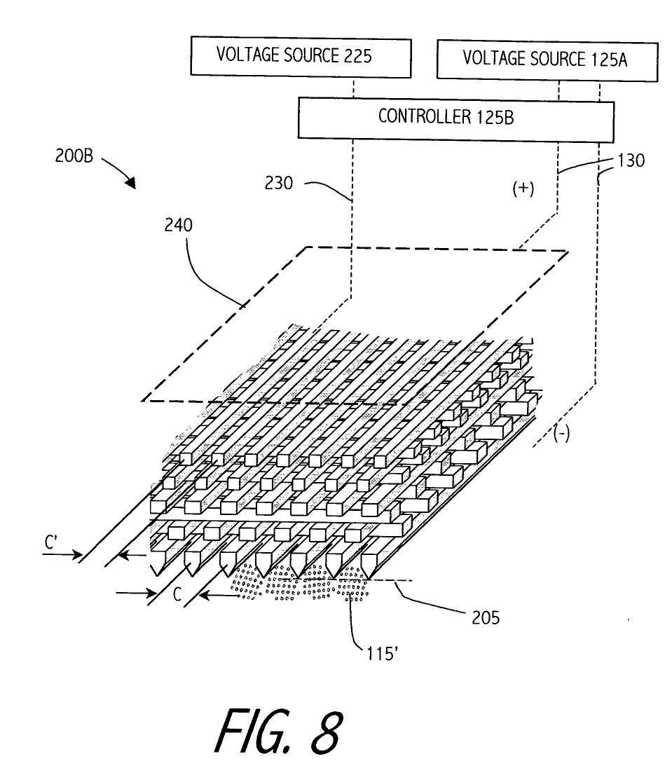

2. Type “B” medical device with refractory lattice. FIG. 7 illustrates an alternative surgical device working end 200A for applying energy to tissue that is similar to that of FIGS. 5 and 6A but differs in several features and methods of operation. FIG. 7 again is a perspective schematic illustration at a highly enlarged scale showing the micron scale lattice structure in working surface 205. The three-dimensional or two-dimension lattice structure 100 is at least partly of a refractory material such as a tungsten alloy that can be resistively heated instantly by electrical current flow therethrough. The refractory lattice portion is indicated at 210a in FIG. 8. The surface lattice layer or layers indicated at 210b are of a substantially electrically insulating material or a metal lattice with an insulative coating 214. The open spatial geometry indicated at 115 of the lattice again is air that can be altered by thermal and/or electric energy into an ionized gas 115′ as illustrated in FIGS. 6A-6B.

As can be seen in FIG. 7, the system has an independent secondary voltage source 225 with first and second leads indicated at 230 (collectively) that are coupled to opposing portions of the lattice for resistively heating the refractory portion 210a. The working end 200A utilizes the first voltage source 125A (an Rf generator) for coupling Rf energy to the plasma 115′ as illustrated in FIGS. 6A and 6B. The first voltage source 125A is operatively coupled to a conductor element 240 that is interior of lattice 100 that will contact the ionized gas 115′ created in the lattice spatial geometry. The controller system 125B optionally has feedback circuitry coupled to impedance and/or temperature sensors for acquiring signals relating to plasma parameters, and controls both electrical sources 125A and 225. In one mode of operation, the secondary voltage source 225 is actuated to instantly create an ionized gas 115′ in the lattice spatial geometry which then floods over a proximate tissue surface T. The first voltage sources 125A then is actuated by the controller to deliver Rf energy to the ionized gas 115′ which serves as an electrode about the insulated surface lattice to delivery energy to tissue. The ionized gas 115′ in the lattice, for example, engages between about 50% and 100% of the tissue surface T, and energy levels can be delivered to the ionized gas electrode to cause surface ablation or deeper heating effects depending on the location of the return electrode or electrodes. If a ground pad is used or a return is spaced substantially apart from the lattice 100, substantial energy density can be created at a selected depth in tissue. If the return is closer about the working end 200B to contact the ionized gas, then very high intensities can be created in the plasma for localized surface ablation.

FIG. 8 illustrates an alternative working end 200B for applying energy to tissue with a lattice having varied dimensional constants C and C′ in different portions of the spatial geometry for confining certain infrared wavelengths in certain regions and allowing infrared emissions in other regions. By this means, it is believed that emissions from surface 205 will create a plasma layer 115′ that extends distally from the working surface. In an underwater surgery, the working surface then can be painted across tissue without firm touching of the tissue to cause surface energy delivery for ablation purposes. It is believed that a controlled and conditioned plasma flow can be created within the lattice by using defects or disorders in the lattice to guide energy particle trajectories.

In another alternative embodiment, the periodic dimensions of the lattice need not be in the range required for confinement and alteration of optical modes in the infrared. The scope of the invention encompasses a refractory lattice that has a substantial open spatial geometry as described above for the creation of an effective gas electrode for use in Rf tissue-contacting surfaces. In one embodiment, the invention consists of a refractory lattice that comprises a heating element wherein the lattice structure has 2D or 3D dimension in the range of about 10 microns or less. In general, the open spatial geometries of a refractory lattice can be adapted for creating a plasma that will assist in controlling energy delivery to tissue.

In another embodiment, the working end can carry a block photonic lattice that defines a plurality of progressive bandgap portions starting in the infrared band together with local lattice defects that allow emissions of a shorter wavelength in a particular direction to the next adjacent bandgap region and local lattice defect to allow progressively shorter wavelength emissions within the block lattice. Ultimately, it is believed, a progressive series of bandgaps and photon-guiding defects will allow mode alterations from longer wavelengths to shorter wavelengths, for example, with ultimate emissions from the working surface in the visible band or an even shorter wavelength. Thus, the scope of the invention includes progressive bandgaps and energy particle guiding lattice portions as known in art for emitting selected wavelengths from a lattice working surface.

In other embodiments, the working end of a probe can carry the lattice of the invention in any form together with other common functionality, such as sterile water or saline irrigation though channels to the lattice, aspiration channels communicating with the working surface, blades and cutting elements adjacent the lattice for sharp dissection of treated tissue and the like.

The term instrument and lattice working surface as used here comprises any instrument for open or endoscopic surgeries for painting across tissue, for pressing against tissue in a selected location, for clamping against tissue as in a jaw structure or for ablating soft tissue, bone, tooth structure, accretions, calculi and the like in any percutanaeous or endoluminal procedure. The working surface can also be carried at the end of a guidewire or catheter for delivering energy to occlusive media in an endovascular procedure.

Those skilled in the art will appreciate that the exemplary systems, combinations and descriptions are merely illustrative of the invention as a whole, and that variations in the dimensions and compositions of invention fall within the spirit and scope of the invention. Specific characteristics and features of the invention and its method are described in relation to some figures and not in others, and this is for convenience only. While the principles of the invention have been made clear in the exemplary descriptions and combinations, it will be obvious to those skilled in the art that modifications may be utilized in the practice of the invention, and otherwise, which are particularly adapted to specific environments and operative requirements without departing from the principles of the invention. The appended claims are intended to cover and embrace any and all such modifications, with the limits only of the true purview, spirit and scope of the invention.

1. An instrument for delivering energy to tissue comprising a working surface at least in part of a three-dimensional photonic lattice.

2. An instrument as in claim 1 wherein the photonic lattice is of a refractory material.

4. An instrument as in claim 1 wherein at least surface portions of the photonic lattice are of an electrical insulator.

5. An instrument as in claim 1 wherein the photonic lattice defines an ordered periodic structure to provide a band gap.

6. An instrument as in claim 1 wherein the photonic lattice defines a disordered periodic structure for guiding photonic energy.

7. An instrument as in claim 2 wherein the photonic lattice defines lattice dimensions for modifying thermal radiation from the working surface.

8. An instrument as in claim 2 wherein the photonic lattice comprises a heating element.

9. An instrument as in claim 1 wherein the photonic lattice defines a plurality of interior spatial regions for acting as diffraction centers for energy particles.

10. An instrument as in claim 9 wherein the spatial regions have ordered uniform dimensions.

11. An instrument as in claim 9 wherein the spatial regions have non-uniform dimensions.

12. An electrosurgical method for applying energy to tissue comprising the steps of:
(a) providing an instrument working surface of a photonic lattice that defines a plurality of spatial regions therein that act as diffraction centers for energy particles; and
(b) causing propagation and controlled diffraction of the energy particles about said spatial regions of the lattice and its working surface to apply energy to proximate tissue.

13. The method as in claim 12 wherein step (b) diffracts energy particles selected from the class consisting of electromagnetic waves, light particles, electrons, ions, microwaves and magnetic waves.

14. The method as in claim 12 wherein step (b) includes the contemporaneous step of heating the photonic lattice.

15. The method as in claim 12 wherein step (b) modifies emissions from a non-preferred mode to a preferred mode.

16. The method as in claim 12 wherein step (b) modifies emissions from a longer wavelength to a shorter wavelength.

17. The method as in claim 12 wherein step (b) includes the contemporaneous step of coupling Rf energy to the energy particles.

18. An instrument for delivering energy to tissue comprising a working surface at least in part of a lattice of a refractory material.

19. An instrument as in claim 18 wherein the lattice defines a 2D or 3D ordered lattice dimensional constant.

20. An instrument as in claim 19 wherein the dimensional constant is less than 10 microns.

21. An instrument as in claim 19 wherein the dimensional constant is less than 5 microns.

22. An instrument as in claim 18 wherein the lattice defines a spatial region that exceeds about 40% of the lattice volume.

23. An instrument as in claim 18 wherein the lattice defines a complete band gap at a selected operative temperature range.

24. An instrument as in claim 23 wherein the band gap is within the infrared band.
